(19) United States
(12) Patent Application Publication
(10) Pub. No.: US 2024/0149238 A1
(43) Pub. Date: May 9, 2024

(54) THERMOPHORETIC CONCENTRATION OF REACTANTS FOR REACTION ACCELERATION

(71) Applicant: Waters Technologies Corporation, Milford, MA (US)

(72) Inventor: Lindsay Morrison, Waltham, MA (US)

(21) Appl. No.: 18/500,612

(22) Filed: Nov. 2, 2023

Related U.S. Application Data
(60) Provisional application No. 63/422,179, filed on Nov. 3, 2022.

(51) Int. Cl. B01J 19/00; G01N 30/02

(52) U.S. Cl. CPC B01J 19/0013; G01N 30/02; B01J 2219/00031; B01J 2219/00054; B01J 2219/00135; B01J 2219/00137; G01N 2030/027

(57) ABSTRACT

The present disclosure discusses a device and method for improving the reaction rate of enzymatic modification of biopolymers. A thermophoretic device is used to increase the rate of enzymatic reactions with biopolymers by creating a temperature gradient using a heating element and a cooling element. The use of a temperature gradient within a cavity in the thermophoretic device concentrates the reacts along an interior surface of the device, increasing the reaction rate.

### CROSS REFERENCE TO RELATED APPLICATIONS

This application claims the benefit of and priority to U.S. provisional patent application Ser. No. U.S. Ser. No. 63/422,179, filed on Nov. 3, 2022, and entitled “Thermophoretic Concentration of Reactants for Reaction Acceleration”; the entire contents thereof is hereby incorporated by reference in its entirety.

### FIELD OF THE TECHNOLOGY

The present disclosure relates to the use of thermophoresis for the acceleration of enzymatic reactions on biopolymers.

### BACKGROUND

Preparative reactions are needed prior to a number of LC-MS assays, particularly assays that are used to analyze biopolymers. Enzymatic reactions are used in many preparative steps for the pre-processing of biopolymers. For example, enzymes can be used to deglycosylate peptides, cleave peptides (e.g., antibodies) and oligonucleotides into smaller fragments, or reduce peptides and oligonucleotides into their constituent monomers (amino acids and nucleic acids, respectively). These enzymatic reactions can be prohibitively slow. For example, a standard digestion of a peptide with trypsin can take from 8 to 24 hours to complete. Solution phase approaches to reaction acceleration tend to lean heavily on temperature and solvent additives, both of which can result in unwanted artifacts.

### SUMMARY

The problems encountered in prior art can be overcome using thermophoresis to create concentration gradients within a thermophoretic device. During a typical enzymatic reaction on a biopolymer, the reaction rate is characterized by an exponential decay in reaction rate as a function of time. This is due to the consumption of reactants (in this case the biopolymer) as the reaction proceeds. The consumption of the biopolymers lowers the concentration of reactants for the enzyme increasing the time it takes for the substrates to bind to each other. A thermophoretic device creates a temperature gradient within the device. Depending on the temperature within the thermophoretic device the reactants move toward the hot side of the temperature gradient, or the cold side of the temperature gradient. The reactants are therefore effectively concentrated in a smaller region of the reaction vessel, which increases the reaction rate.

In an embodiment, a thermophoretic device comprises: a heating element, and a cooling element, wherein the heating element and the cooling element together define a cavity between the heating element and the cooling element. The thermophoretic device also includes a temperature controller coupled to the heating element and the cooling element. The temperature controller is configured to regulate the temperature of the heating element and the cooling element such that the heating element is kept at a higher temperature than the cooling element.

In one embodiment, the heating element is positioned above the cooling element. The heating element can be separable from the cooling element. A cavity is formed when the heating element is placed on the cooling element. In some embodiments, the heating element comprises a convex surface and the cooling element comprises a concave indentation. When the heating element and the cooling element are in contact with each other, a substantially bowl-shaped cavity is formed between the heating element and the cooling element. The cavity can have a volume of between about 25 μL and 250 μL. The cavity can have a thickness of between about 2 μm and 50 μm.

The heating element, in some embodiments, is a resistive heater (e.g., a cartridge heater). The cooling element can be a Peltier device. The heating element and the cooling element can be composed of stainless steel.

The temperature controller monitors and controls the temperature of the heating and cooling elements during use. The temperature controller is configured to maintain the temperature of the heating element at a temperature between about 20° C. and about 40° C. The temperature controller is also configured to maintain the temperature of the cooling element at a temperature between about 15° C. and about 5° C.

The thermophoretic device, as described herein, can be used to increase the rate of enzymatic modification of a biopolymer. In an embodiment, a method of enzymatic modification of a biopolymer comprises: forming a mixture of the biopolymer with an enzyme that modifies at least a portion of the biopolymer; adding the mixture to a thermophoretic device as described herein; increasing the temperature of the heating element to a temperature of about 30° C. to about 40° C.; and decreasing the temperature of the cooling element to a temperature of about 5° C. to about 15° C. By creating a temperature gradient within the thermophoretic device, the biopolymer and enzymes are attracted to a surface of the device, effectively increasing the concentration of the reagents.

In an embodiment, the concentration of the biopolymer in the mixture is about 1 mg/mL to about 5 mg/mL. In an embodiment, the concentration of the enzyme in the mixture is about 0.01 mg/mL to about 1 mg/mL.

The thermophoretic enhanced method described herein can be used on biopolymers such as polypeptides, polynucleotide, and polysaccharides. Exemplary biopolymers include proteins, antibodies, DNA and RNA. The enzymes used in the described method include enzymes that modify the biopolymer by digestion of the biopolymer. Enzymatic digestion of the biopolymer breaks down a biopolymer into smaller fragments. Exemplary enzymes that can be used in the disclosed method include serine protease (e.g., trypsin), glycosidase, and restriction enzymes.

In a typical procedure a mixture of the biopolymer and the enzyme is placed in a thermophoretic device having a defined cavity. When the heating element is placed onto the cooling element, the mixture is dispersed within the cavity as a thin film. The thickness of the film is between about 2 μm and 10 μm. The cavity can have a volume of between about 25 μL and 250 μL. The heating element and cooling element are set at the appropriate temperatures for the thermophoretic reaction, and the reaction is allowed to proceed for a predetermined time. Once the reaction is complete, or a sufficient amount of time has passed, the sample is analyzed using liquid chromatography (e.g., high pressure liquid chromatography.

### BRIEF DESCRIPTION OF THE DRAWINGS

The technology will be more fully understood from the following detailed description taken in conjunction with the accompanying drawings, in which:

FIG. 1 depicts a schematic diagram of an enzymatic reaction of an antibody.

FIG. 2 is a schematic diagram of a thermophoretic device.

FIG. 3 is a schematic diagram of a heating element.

FIG. 4 is a schematic diagram of a cooling element.

FIG. 5 is a side view of a heating element and cooling element having convex and concave surfaces, respectively.

FIG. 6 is a bar graph depicting the relative abundance of peptide fragments obtained by thermophoretic (10° C.-30° C.) and isothermal (20° C. or 30° C.) trypsin enzyme digestions.

FIG. 7A, FIG. 7B, FIG. 7C, and FIG. 7D depict line graphs of the relative abundance of peptide fragments over time obtained by thermophoretic (10° C.-30° C.) and isothermal (20° C. or 30° C.) trypsin enzyme digestions.

FIG. 8 is a bar graph depicting the relative abundance of peptide fragments obtained by thermophoretic (28° C.-40° C.), thermophoretic (10° C.-30° C.), and isothermal (28° C. or 40° C.) trypsin enzyme digestions.

FIG. 9 is a bar graph is a bar graph depicting the relative abundance of peptide fragments obtained by thermophoretic (10° C.-35° C.), isothermal film (35° C.), or isothermal open vessel (35° C.).

FIG. 10A and FIG. 10B shows kinetic plots for reaction yield using partially reduced and natively folded proteins.

FIG. 11 shows a bar graph comparing yield gains between thermophoretic films and isothermal films for different peptide states.

### DETAILED DESCRIPTION

To overcome the problems of prior sample preparation methods, a thermophoretic device and method was designed to improve the reaction kinetics for enzymatic modification of a biopolymer. The novel device and method use a temperature gradient to concentrate the reactants, thereby increasing the reaction rate.

Thermophoresis is a phenomenon in which molecules move in solution as a consequence of a temperature gradient. The strength and directionality of this relationship can be described by the Soret coefficient. The Soret coefficient is defined as DT/D, where DT is the thermodiffusion coefficient and D is the ordinary diffusion coefficient. The thermophoretic strength and direction is influenced by a number of factors including, but not limited to, electrolyte composition and concentration, analyte size, temperature difference, average temperature, and analyte composition.

The thermodiffusion coefficient is a proportionality constant linearly relating drift velocity V to the temperature gradient ΔT. Consequently, the thermodiffusion coefficient can be given by ST×D, where ST is the Soret coefficient and D is the ordinary diffusion coefficient. For ST>0, reactants move toward the colder temperature. For ST<0, reactants move toward the warmer temperature. One unique characteristic of thermophoresis is that the Soret coefficient strongly depends on average temperature and features a temperature, the Soret temperature (T*), at which analytes change from being thermophilic (moving toward the hot side) to thermophobic (moving toward the cold size). In solutions above 20° C., most biopolymers are positively thermophoretic and drift towards the cooler temperature regions. The time that it takes for the biopolymers to move to the cooler temperature regions is distance dependent.

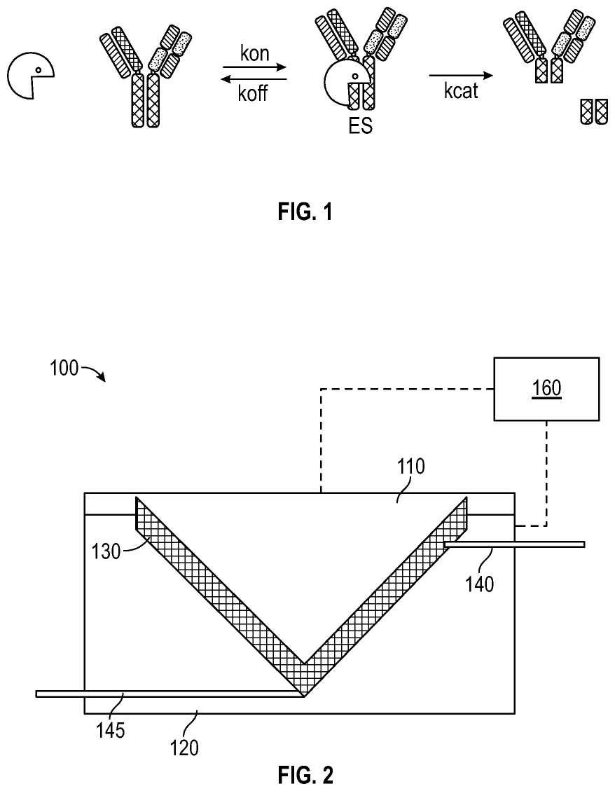

FIG. 1 depicts a schematic diagram of an enzymatic reaction of an antibody. The reaction rate is controlled by the time it takes for the enzyme to bind to the substrate (antibody). This is defined by the rate constant kₒₙ. k_{off} is the dissociation rate constant for the enzyme with respect to the substrate. k_{cat} is the rate constant for the enzymatic reaction with the substrate. In a typical enzymatic reaction k_{off} is slow and k_{cat} is fast. The rate controlling step is therefore the binding of the enzyme to the substrate. As the substrate is depleted through the enzymatic reactions, the rate reaction rate, km slows, since the time it takes for the substrate and the enzyme to diffuse and bind becomes longer with less substrate available.

In one embodiment of thermophoretic assisted enzymatic reactions, the reaction is run in a vessel having a heated section and a cooled section. The temperature differential creates a concentration of reactants along either the cooled section of the vessel or the heated section of the vessel. The location of the concentration of reactants is based on the Soret temperature and the average temperature of the reaction. For enzymatic reactions of biopolymers, the average temperature of the reaction is kept between 10° C. and 35° C. to prevent damage to the biopolymers that would create unintentional artifacts in the subsequent analysis. Concentration of the reactants along the interior surface of the cavity improves the diffusional limitations associated with kₒₙ and thereby accelerates the reaction. Acceleration of the reaction can be further improved by repeated mixing and subsequent application of a temperature gradient to the reaction vessel.

FIG. 2 depicts a schematic view of a thermophoretic device 100. Thermophoretic device 100 includes a heating element 110 and a cooling element 120. Heating element 110 and cooling element 120, together, define a cavity 130. A temperature controller 160 is coupled to the heating element and the cooling element. During use, the temperature controller provides control signals to both the heating element and the cooling element to regulate the temperature of the heating element and the cooling element. During use the temperature of the heating element is kept at a higher temperature than the cooling element.

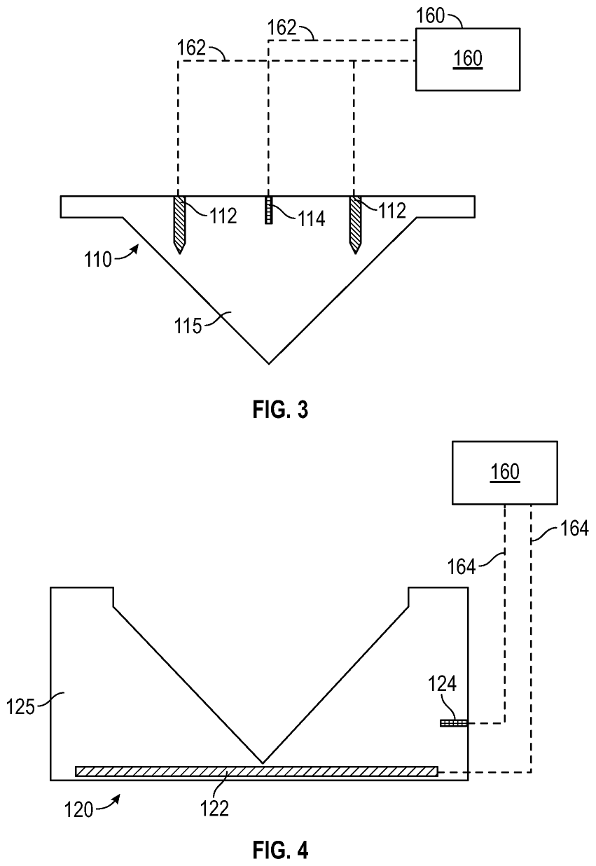

Heating element 110 can be any device capable of maintaining an interior surface within the defined cavity at a temperature of, at least, between about 20° C. and 40° C. FIG. 3 depicts a schematic diagram of a heating element 110. In a preferred embodiment, the heating element is composed of a thermally conductive body 115. The body should also be composed of a material that will not react with the biopolymers or enzymes being used during the reactions. A preferred material for the body is stainless-steel. Heating is provided to the body using one or more resistive heaters 112 coupled to the body. For example, one or more cartridge resistive heaters can be positioned within the body to provide heat to the body. A thermistor 114 can also be coupled to the body. The temperature controller 160 is electrically coupled to the resistive heaters and the thermistor through control lines 162. During use the temperature controller can monitor the temperature of the heating element through the thermistor and adjust the temperature of the heating element by providing control signals and/or an electrical current to the heating elements.

Cooling element 120 can be any device capable of maintaining an interior surface within the defined cavity at a temperature of, at least, between about 5° C. and 15° C. FIG. 4 depicts a schematic diagram of a cooling element. In a preferred embodiment, the heating element is composed of a thermally conductive body 125. The body should also be composed of a material that will not react with the biopolymers or enzymes being used during the reactions. A preferred material for the body is stainless-steel. Heating is provided to the body using one or more cooling devices 122 coupled to the body. For example, a Peltier cooling plate can be positioned within the body to cool the body. A temperature sensor 124 can also be coupled to the body and/or can be incorporated in the cooling device. The temperature controller 160 is electrically coupled to the cooling device 122 and the temperature sensor through control lines 164. During use the temperature controller can monitor the temperature of the cooling element through the temperature sensor and adjust the temperature of the cooling element by providing control signals and/or an electrical current to the cooling elements.

In an aspect of the device, the heating element is positioned above the cooling element. The heating element may be separable from the cooling element. In this aspect, the heating element can be lifted away from the cooling element. This allows access to the cavity that is formed when the heating element is brought back into contact with the cooling element.

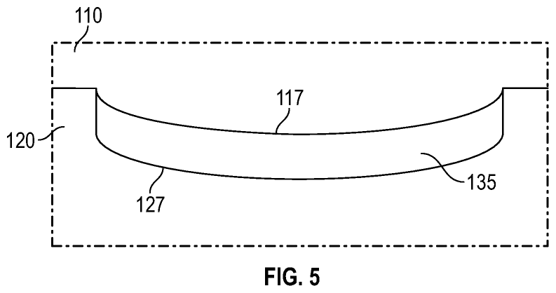

In an aspect of the device, the heating element 110 has a convex surface 117, and the cooling element 120 has a concave surface 127, as shown in FIG. 5. When the heating element and the cooling element are in contact with each other, a substantially bowl-shaped cavity 135 is formed between the convex surface of the heating element and the concave surface of the cooling element. Referring to FIG. 2, the heating element can have a conical surface that is complementary with a conical surface of the cooling element. When the heating element and cooling element are in contact with each other, a substantially conical cavity is formed between the heating element and the cooling element.

While the thermophoretic device is not limited to any size, it is preferred that the device has a size that allows the reactants to migrate during a thermophoretic enhanced reaction at a rate that allows the reaction rate to be accelerated. For example, if the distance between the heating element and the cooling element is too great, the time for the reactants to migrate to the hot or cold side will be too great for a noticeable increase in reaction rate to occur. As discussed above, when the heating element and cooling elements are assembled, a cavity is formed between the heating element and the cooling element. The distance between the exterior surfaces of the heating element and the cooling element that define the cavity is herein referred to as the cavity thickness. For enzymatic reactions of biopolymers, when the thermophoretic device is assembled, the thickness of the cavity is between 2 μm and 10 μm. For enzymatic reactions of biopolymers, the preferred the volume of the cavity is between 25 μL and 250 μL.

During use a liquid mixture is added to the bowl-shaped cavity. The heating element is brought down onto the cooling element to seal the cavity. When the heating element is brought into contact with the cooling element, the mixture is dispersed within the cavity as a thin film. The thickness of the film is equivalent to the cavity thickness, between about 2 μm and 10 μm.

The thermophoretic device can be used to enhance the rate of enzymatic reactions with biopolymers. In one embodiment, an enzymatic modification of a biopolymer is performed in the thermophoretic device. As used herein a biopolymer is a polymer produced by the cells of living organisms. Biopolymers are composed of monomeric units that are covalently bonded to form larger molecules. Exemplary biopolymers include, but are not limited to, polynucleotides (e.g., DNA, RNA, mRNA, etc.), polypeptides (e.g., proteins and antibodies), and polysaccharides (e.g., starch, cellulose and alginate).

In some analytical methods it is desirable to modify biopolymers before performing a chromatographic analysis. For example, some analytic methods require digestion of the biopolymer. Digestion of a biopolymer can be performed enzymatically using an appropriate enzyme. For polypeptides, enzymatic digestion will break down a polypeptide into smaller fragments, sometimes referred to as oligopeptides. Similarly, enzymatic digestion of polynucleotides with produce smaller fragments known as oligonucleotides. Enzymatic digestion of polysaccharides can be used to produces saccharide fragments.

Polypeptides can be modified using enzymes prior to analysis. For example, serine proteases can be used to break a polypeptide into smaller fragments. A serine protease cleaves peptide bond at serine amino acids. Serine proteases include trypsin, chymotrypsin, elastase, subtilisin, IGdE, IdeS, and IdeZ proteases, with each class of protease having a specific cleavage site on the polyprotein. Polypeptides can also be modified using a glycoside enzyme. A glycosidase enzyme can be used to deglycosylate N-linked glycans on the polypeptides in the sample. An exemplary glycosidase enzymes is PNGaseF.

Polynucleotides can be modified with enzymes prior to analysis. In one aspect of the disclosure, a polynucleotide can be broken into fragments (e.g., oligonucleotides) using restriction enzymes. Restriction enzymes cleave polynucleotides at sequence-specific sites producing oligonucleotides with a known sequence at each end.

The enzymatic modification of a biopolymer can be accelerated under thermophoretic reaction conditions. A mixture of the biopolymer and an enzyme is formed. The enzyme is chosen for the specific modification of the biopolymer that is desired. The concentration of the biopolymer in the mixture is about 1 mg/mL to about 5 mg/mL. The concentration of the enzyme in the mixture is about 0.01 mg/mL to about 1 mg/mL.

The mixture is added to a thermophoretic device (e.g., the thermophoretic device described herein). The thermophoretic device includes a heating element and a cooling element. To increase the reaction rate of the enzymatic modification of the biopolymer, the heating element is heated to a temperature of about 30° C. to about 40° C., while the cooling element is cooled to about 5° C. to about 15° C. The difference in temperature between the heating element and the cooling element creates the thermophoretic conditions that draw the reactants toward one or the elements, increasing the reaction rate of the enzymatic modification. In an embodiment, the difference in temperature between the heating element and the cooling element, ΔT, is between about 15° C. and about 35° C.

The method of thermophoretic acceleration of a reaction can be performed as a batch reaction or in a semi-continuous operation. In a batch operation, the mixture is disposed in the cavity a thermophoretic device. The heating element and the cooling element of the thermophoretic device are initially at about room temperature (e.g., about 20° C. to about 25° C.). Once the mixture is positioned within the thermophoretic device, the temperature of the heating element is increased, while the temperature of the cooling element is decreased. The heating element is heated to a temperature of about 30° C. to about 40° C., while the cooling element is cooled to about 5° C. to about 15° C. Under these conditions the reactants are drawn toward the cooling plate and are concentrated, increasing the reaction rate.

FIG. 2 depicts a thermophoretic device that is capable of being operated in a semi-batch mode. In a semi-batch reaction mode, a sample is introduced into the thermophoretic device. In one aspect, the thermophoretic device includes an inlet 140 and an outlet 145. In a semi-batch operation, a mixture of a biopolymer and enzyme is introduced in the reaction cavity 130, through the inlet 140. During the introduction of the mixture, the heating element and the cooling element are at the same, or substantially similar, temperatures. Typically, during the introduction step, the top and bottom elements are kept at room temperature.

After the mixture is introduced into the thermophoretic device, the device is operated as in the batch method. The temperature of the heating element is increased, while the temperature of the cooling element is decreased. The heating element is heated to a temperature of about 30° C. to about 40° C., while the cooling element is cooled to about 5° C. to about 15° C. Under these conditions the reactants are drawn toward the heating element or cooling element and are concentrated, increasing the reaction rate. Under typical enzymatic reaction conditions, the reactants are drawn toward the cooling element.

Once the enzymatic reaction is completed, the mixture containing the resulting biopolymer fragments is removed from the thermophoretic device through outlet 145. After removal of substantially all of the sample from the thermophoretic device, inlet 140 was used to introduce the next mixture into the reactant.

After the completion of the enzymatic reaction under thermophoretic conditions, the sample is introduced into a chromatography system for analysis. In a preferred aspect of the disclosure, a high-pressure liquid chromatography (HPLC) system is used for the analysis.

### EXPERIMENTAL

### Example 1

Trypsin is an enzyme that selectively cleaves proteins specifically at lysine and arginine. To test the effects of thermophoretic reaction conditions on enzymatic reactions a polypeptide was treated with trypsin under isothermal and thermophoretic conditions. The reaction is quenched and the sample was analyzed using HPLC-MS. The relative abundance of oligopeptide residues was compared to determine the effectiveness of the thermophoretic process.

A polypeptide was mixed with trypsin to test the use of thermophoretic reaction conditions to increase the rate of trypsin digestion of the polypeptide. A mixture of a mAb (12.5 μL of 2 mg/mL) and trypsin (12.5 μL of 0.1 mg/mL) was formed. The mixture was placed in a thermophoretic device as depicted in FIG. 2. In the first test, the mixture was treated under isothermal conditions at 20° C. In the second test the mixture was treated under isothermal conditions at 30° C. In the third test the mixture was treated under thermophoretic conditions, where the heating element was held at 30° C. and the cooling element was held at 10° C. Each of the tests was run for 20 minutes. At the end of the 20 minutes, the enzymatic reaction was quenched by the addition of 200 uL of 6 M guanidine hydrochloride. The sample is removed from the thermophoretic device and injected into an HPLC-MS chromatography system.

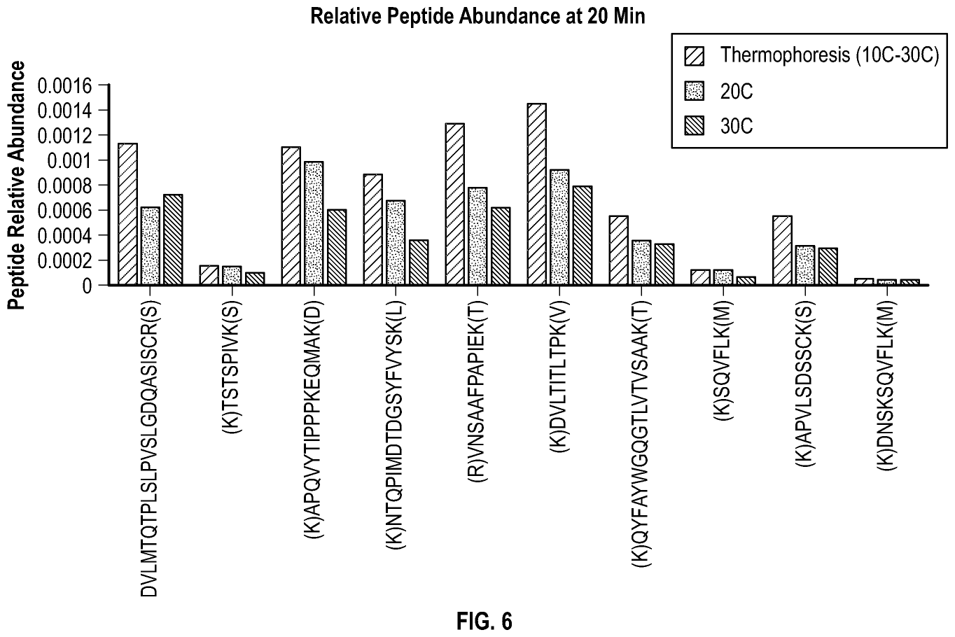

FIG. 6 shows the relative abundance of oligopeptides produced by trypsin digestion of the polypeptide. The relative abundance of the oligopeptides was significantly greater when thermophoretic conditions were used to digest the polypeptide, when compared to both 20° C. and 30° C. isothermal conditions.

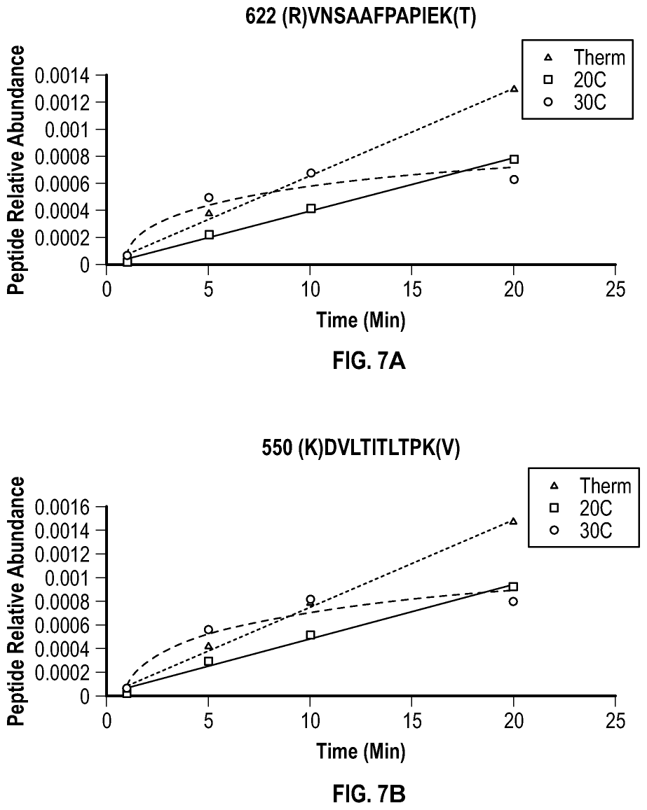

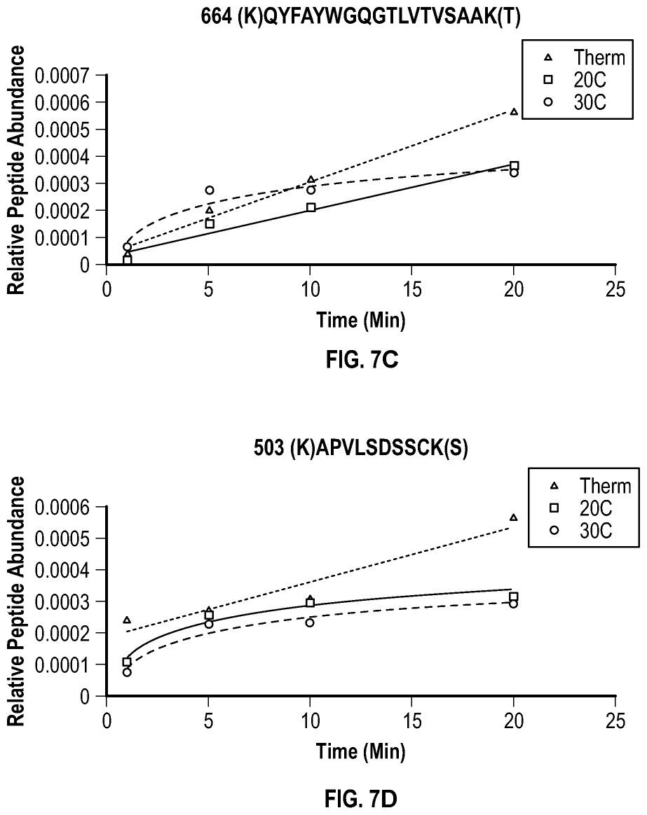

Each of FIG. 7A, FIG. 7B, FIG. 7C and FIG. 7D shows a graph of the relative abundance of selected oligopeptides over a time period of 20 minutes. Aliquots from the three tests were removed and analyzed using HPLC at 5, 10, 15, and 20 minutes. For the first 10 minutes, the rate of reaction between the thermophoretic conditions (10-30° C.) and isothermal (30° C.) were similar. After 10 minutes, the rate of reaction of the isothermal (30° C.) reaction slows significantly, while the rate of reaction under thermophoretic conditions remains the same. The thermophoretic experiments demonstrate linear reaction yields over time. In comparison, the isothermal reaction was shown to have logarithmic yields due to depletion of the reactants.

### Example 2

In another experiment, the same polypeptide was digested using trypsin under thermophoretic conditions and isothermal conditions. Both the temperature of the heating element and the cooling element were elevated to see if a further increase in the rate of trypsin digestion could be obtained through the use of higher temperatures. In this experiment, the same mixture of polypeptide and trypsin was used as discussed in Example 1. In the first test, the mixture was treated under isothermal conditions at 28° C. In the second test the mixture was treated under isothermal conditions at 40° C. In the third test the mixture was treated under thermophoretic conditions, where the heating element was held at 30° C. and the cooling element was held at 10° C. In the fourth test the mixture was treated under thermophoretic conditions, where the heating element was held at 48° C. and the cooling element was held at 28° C. Each of the tests was run for 20 minutes. At the end of the 20 minutes, the enzymatic reaction was quenched by the addition of 200 uL of 6 M guanidine hydrochloride. The sample is removed from the thermophoretic device and injected into an HPLC-MS chromatography system.

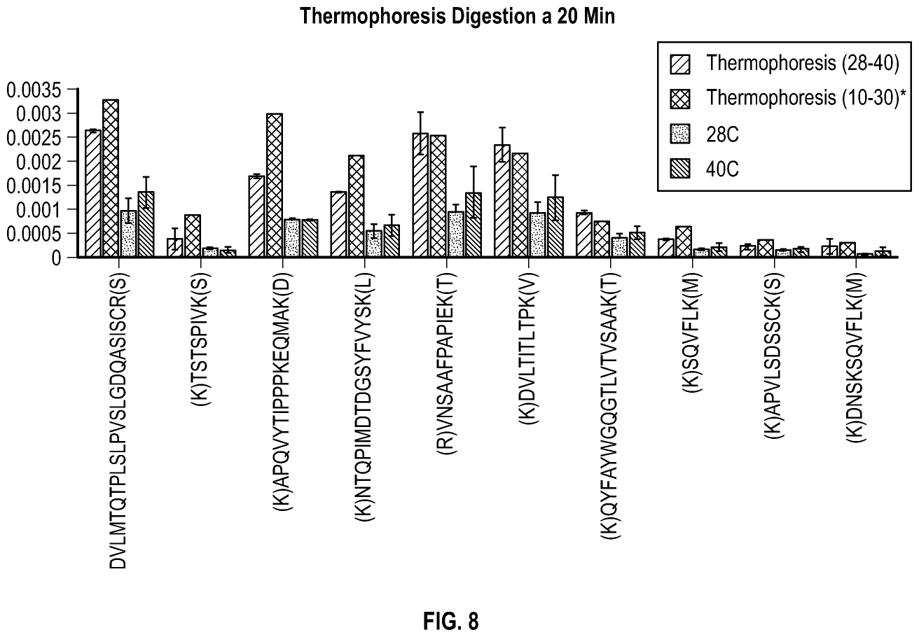

FIG. 8 shows the relative abundance of oligopeptides produced by trypsin digestion of the polypeptide. The relative abundance of the oligopeptides was significantly greater when thermophoretic conditions were used to digest the polypeptide, when compared to both 28° C. and 40° C. isothermal conditions. Both the high temperature thermophoretic conditions (28-40° C.) and the low temperature thermophoretic conditions (10-30° C.) behave similarly, indicating that the undigested material moves toward the high temperature side in both cases. Thermophoresis outperforms digestion at the higher isothermal temperature (40° C.) by nearly double. This is an important finding given that higher temperatures used during isothermal processing can induce artifacts in the sample.

### Example 3

In this experiment three tests were performed. In the first test the mixture from Example 1 was digested with trypsin at 35° C. in an open vessel. In the second test, the mixture from Example 1 was placed in the apparatus depicted in FIG. 2 under isothermal conditions (35° C.). In the second test the mixture was heated as a thin film between the heating element and the cooling element. In the third test the mixture was subjected to thermophoretic conditions (10° C. cooling element, 35° C. heating element). The mixture was treated as a thin film in the third test. In all of these tests the substrate polypeptide was partially reduced but natively folded.

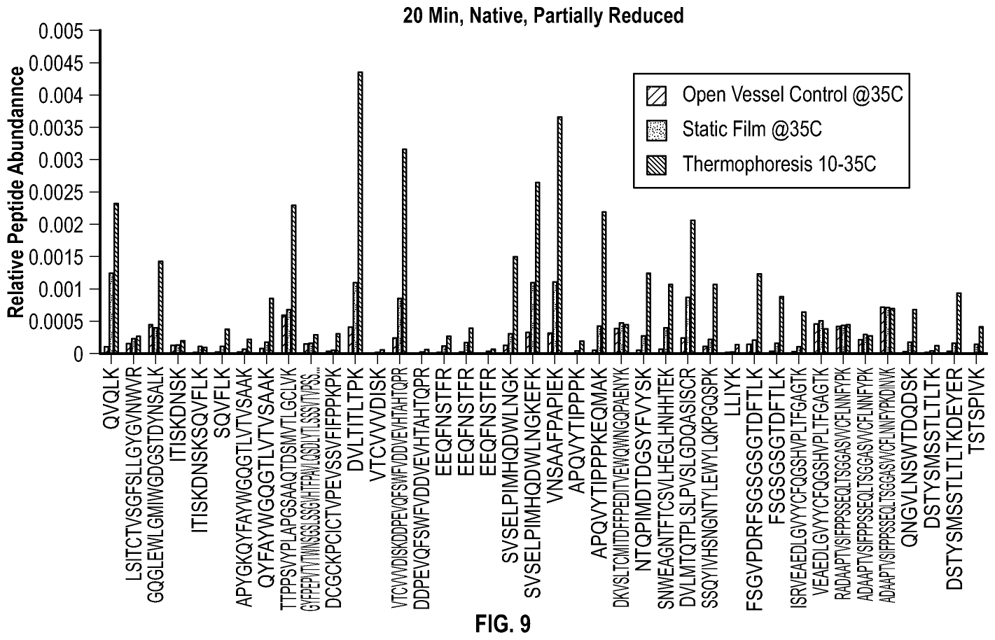

FIG. 9 shows the relative abundance of oligopeptides produced by trypsin digestion of the polypeptide. The relative abundance of the oligopeptides was significantly greater when thermophoretic conditions were used to digest the polypeptide, when compared to both isothermal conditions: static film at 35° C. and open vessel control at 35° C.

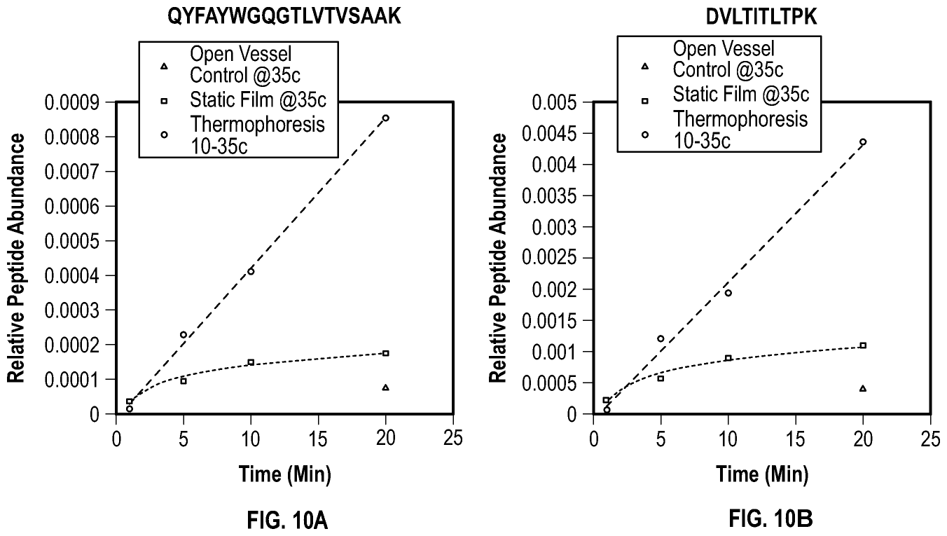

FIG. 10A and FIG. 10B show a graph of the relative abundance of selected oligopeptides over a time period of 20 minutes for these tests. Aliquots from the three tests were removed and analyzed using HPLC at 5, 10, 15, and 20 minutes. After 5 minutes, the rate of reaction of the isothermal (35° C.) reaction slows significantly, while the rate of reaction under thermophoretic conditions remains the same. The thermophoretic experiments demonstrate linear reaction yields over time. In comparison, the isothermal reaction was shown to have logarithmic yields due to depletion of the reactants.

### Example 4

In this test the yield of oligopeptides produced from a polypeptide in different states was investigated. In the first set of experiments denatured polypeptide was digested with trypsin at isothermal conditions (30° C.) and thermophoretic conditions (10° C. cooling element, 30° C. heating element). In the second set of experiments partially reduced polypeptide was digested with trypsin at isothermal conditions (30° C.) and thermophoretic conditions (10° C. cooling element, 30° C. heating element). In the third set of conditions partially reduced, natively folded polypeptide was digested with trypsin at isothermal conditions (35° C.) and thermophoretic conditions (10° C. cooling element, 35° C. heating element).

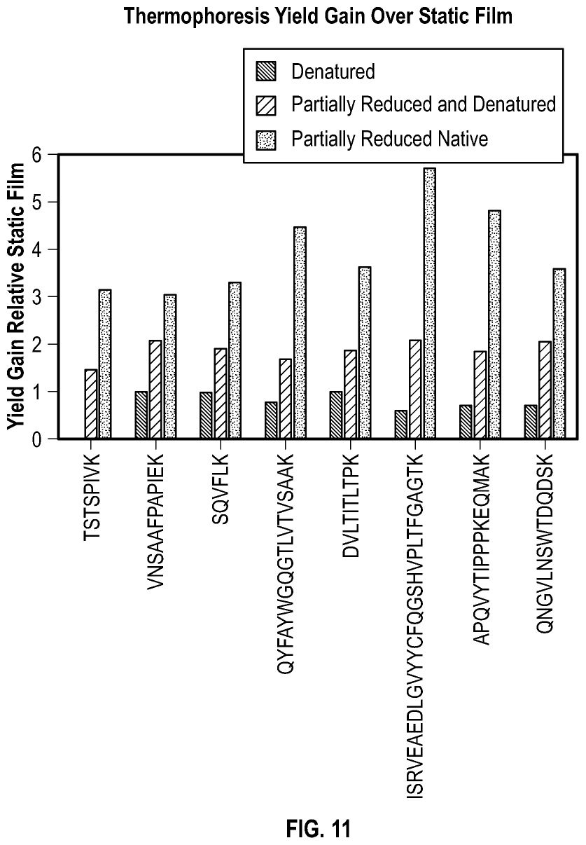

FIG. 11 shows the yield of the given oligopeptides relative to their yield in the under isothermal conditions, Abund-thermophoretic/Abund-isothermal. The results show significant yield gains for each of the different states of the polypeptides. The greatest increase in yield occurs for the partially reduced, natively folded peptide.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A thermophoretic device comprising:
a heating element;
a cooling element;
wherein the heating element and the cooling element together define a cavity between the heating element and the cooling element; and
a temperature controller coupled to the heating element and the cooling element, wherein the temperature controller is configured to regulate the temperature of the heating element and the cooling element, such that the heating element is kept at a higher temperature than the cooling element.

2. The thermophoretic device of claim 1, wherein the heating element is positioned above the cooling element.

3. The thermophoretic device of claim 1, wherein the heating element is separable from the cooling element, and wherein the cavity is formed when the heating element is placed on the cooling element.

4. The thermophoretic device of claim 1, wherein the heating element comprises a convex surface and wherein the cooling element comprises a concave indentation, wherein when the heating element and the cooling element are in contact with each other, a substantially bowl-shaped cavity is formed between the heating element and the cooling element.

5. The thermophoretic device of claim 1, wherein the cavity has a volume between about 25 μL to about 250 μL.

6. The thermophoretic device of claim 1, wherein the cavity has a thickness of between about 2 μm and 10 μm.

7. The thermophoretic device of claim 1, wherein the heating element comprises a resistive heater.

8. The thermophoretic device of claim 1, wherein the cooling element comprises a Peltier device.

9. The thermophoretic device of claim 1, wherein the heating element and the cooling element are composed of stainless steel.

10. The thermophoretic device of claim 1, wherein the temperature controller is configured to maintain the temperature of the heating element at a temperature of between about 20° C. to about 40° C.

11. The thermophoretic device of claim 1, wherein the temperature controller is configured to maintain the temperature of the cooling element at a temperature of between about 15° C. to about 5° C.

12. A method of enzymatic modification of a biopolymer comprising:
forming a mixture of the biopolymer with an enzyme that modifies at least a portion of the biopolymer;
adding the mixture to a thermophoretic device comprising a heating element and a cooling element;
increasing the temperature of the heating element to a temperature of about 30° C. to about 40° C.; and
decreasing the temperature of the cooling element to a temperature of about 5° C. to about 15° C.

13. The method of claim 12, wherein the concentration of the biopolymer in the mixture is about 1 mg/mL to about 5 mg/mL.

14. The method of claim 12, wherein the concentration of the enzyme in the mixture is about 0.01 mg/mL to about 1 mg/mL.

15. The method of claim 12, wherein the biopolymer is a polypeptide.

16. The method of claim 12, wherein the biopolymer is a protein.

17. The method of claim 12, wherein the biopolymer is an antibody.

18. The method of claim 12, wherein the enzyme is a serine protease.

19. (canceled)

20. (canceled)

21. The method of claim 12, wherein the biopolymer is a polynucleotide.

22. (canceled)

23. (canceled)

24. The method of claim 21, wherein the enzyme is a restriction enzyme.

25. The method of claim 12, wherein adding the mixture to the thermophoretic device comprises placing the mixture in a cavity defined by the heating element and the cooling element.

26. The method of claim 25, wherein the mixture is added to the cooling element and wherein the heating element is moved toward the cooling element to disperse the mixture in the cavity as a film.

27. (canceled)

28. (canceled)

29. (canceled)

30. The method of claim 12, wherein a difference in temperature between the heating element and the cooling element is between about 15° C. and about 35° C.

31. The method of claim 12, further comprising analyzing the enzymatically modified biopolymer sample using liquid chromatography.
